(19) United States
(12) Patent Application Publication
(10) Pub. No.: US 2010/0004711 A1
(43) Pub. Date: Jan. 7, 2010

(54) MEDICAL IMPLANT

(75) Inventor: Andreas Hahn, Berg (DE)

Correspondence Address: WOLF GREENFIELD & SACKS, P.C., 600 ATLANTIC AVENUE, BOSTON, MA, 02210-2206, US

(73) Assignee: Sorin Group Deutschland GmbH, Munchen (DE)

(21) Appl. No.: 12/493,854

(22) Filed: Jun. 29, 2009

(30) Foreign Application Priority Data
Jul. 2, 2008 (EP) 08 159 538.1

(51) Int. Cl. A61N 1/375; A61N 1/378; A61N 1/39

(52) U.S. Cl. 607/5; 607/36; 607/35

(57) ABSTRACT

A medical implant, in particular a pacemaker, cardioverter, defibrillator or the like, having at least one functional unit (2, 4), such as, for example, an energy storage (2), control electronics (4) or a combination thereof, and a housing (20) for accommodating the at least one functional unit (2, 4). The medical implant furthermore comprises at least one electrode (10), having a connection end (12), an electrode lead (11) and a stimulation end (13), the connection end (12) of the electrode (10) being arranged in the housing (20) for accommodating the at least one functional unit (2, 4).

### FIELD OF THE INVENTION

The invention relates to a medical implant, in particular a cardiac implant such as, for example, a pacemaker, cardioverter, defibrillator or the like, as known from, for example, U.S. Pat. No. 6,327,502, on which the preamble of claim 1 is based.

### PRIOR ART

Known medical implants, such as, for example, pacemakers, cardioverters or defibrillators, normally comprise a fluid-tight, i.e. liquid-tight and gas-tight, housing. At least one set of control electronics and a long-lasting energy storage for supplying the electronics are accommodated in the housing. Furthermore, a terminal housing (header) having standardised connection sockets for connecting electrodes is disposed on the housing. The connection sockets have contacts for electrically connecting the electrodes with the control electronics inside the housing.

Such a medical implant is schematically shown in FIG. 1. As is apparent from FIG. 1, the medical implant comprises a housing 120, arranged in which are the control electronics 104 and an energy storage 102. Furthermore, arranged on the top side of the housing 120 is a terminal housing 130 having connection sockets 114 for electrically connecting electrodes 110 with the control electronics 104 inside the housing. In order to create such an electrical connection, a respective connection end (not shown) of an electrode lead 111 is inserted into the connection socket 114. Provided at the opposite end of the electrode lead 111 to the connection end is a stimulation end 113, which is to be placed in the heart muscle of a patient. The housing 120 and the terminal housing 130 together form a fluid-tight housing arrangement, with the control electronics 104 inside the housing 120 being connected via leads with contacts (not shown) of the connection sockets 114. For assembly of the terminal housing 130 on the housing 120, special bushings (not shown) are generally used for fluid-tight electrical connection.

High quality requirements are placed on known medical implants. In particular, the housing and the terminal housing must together form a fluid-tight housing arrangement for a period of, in general, about ten years in order to ensure the functionality of the implant and rule out any danger to the patient's health. For this purpose, a plug-and-socket connection of the electrodes on the terminal housing, which remains fluid-tight for years, is also required. Furthermore, this plug-and-socket connection must also withstand a permanent dynamic load that is caused by movements of the patient. So as to remain fluid-tight under the specified circumstances over the entire service life, the terminal housing and the bushings used for fluid-tight electrical connection have an accordingly complex structure, which contributes to high manufacturing costs of the medical implant. Owing to its complex structure, the terminal housing takes up a relatively large part, in general approximately ⅓, of the overall dimension of the medical implant.

### DESCRIPTION OF THE INVENTION

It is therefore the object of the present invention to provide a medical implant which has a smaller overall dimension and is inexpensive to produce.

This object is solved according to the invention by means of a cardiac implant according to claim 1.

According to one aspect of the present invention, a medical implant, in particular a pacemaker, cardioverter, defibrillator or the like, is provided, which has at least one functional unit, such as, for example, an energy storage, control electronics or a combination thereof; a housing for accommodating the at least one functional unit; and at least one electrode having a connection end, an electrode lead and a stimulation end. In the medical implant, the connection end of the electrode is arranged in the housing for accommodating the at least one functional unit.

Owing to the arrangement according to the invention of the connection end of the electrode directly in the housing for accommodating the at least one functional unit, a separate terminal housing can be dispensed with. This reduces the overall dimension of the medical implant by approximately ⅓ as compared to corresponding implants of the prior art. Furthermore, owing to the design according to the invention, no separate bushings for creating a fluid-tight electrical connection between the housing and the terminal housing are necessary. Since neither a terminal housing that is complex to manufacture nor electric bushings are necessary, the costs for manufacturing the medical implant are also reduced.

According to the invention, the medical implant is designed in the form of a cardiac implant such as, for example, a pacemaker, cardioverter or defibrillator.

At least one functional unit, such as, for example, an energy storage, control electronics or a combination thereof, is accommodated in the housing of the medical implant. Preferably at least two functional units in the form of an energy storage and control electronics are accommodated in the housing. The energy storage accommodated in the housing supplies direct current that is converted into medically useable pulses or fields by the control electronics, which are then transmitted via the electrodes to the patient's heart muscle.

The medical implant comprises at least one, preferably two to four electrodes, the respective connection ends of which are directly arranged in the housing for accommodating the at least one functional unit. According to the invention, the connection end of the electrode is attached directly to the corresponding functional unit, for example the control electronics. The direct connection of the connection end of the electrode to the control electronics preferably occurs by means of bonding or a soldered connection, both of which are a type of connection that is inexpensive and easy to carry out.

The medical implant is preferably read out using telemetry and programmed by external devices. Telemetry, or also remote measurement, hereby relates to the transmission of measured values of a measuring element (sensor) located at the measurement site to a spatially separate location. In the case of programming using external devices, data from a first device is transmitted to a spatially separate second device, with the data comprising program instructions to be carried out by the second device. For example, the reprogramming or the retrieval of data of the medical implant takes place with the aid of a programming device having a programming head that is placed on the patient's skin above the position of the medical implant. The programming head comprises a station with a loading function for reading out and programming data. Parameters as well as diagnostic values of the medical implant are thereby transmitted and evaluated. Finally, the updated parameters are loaded back into the medical implant. Thus, a further operation on the patient is not necessary for the function control or reprogramming of the medical implant.

According to a further development of the invention, the housing is formed as a single part. In this case, the housing is formed directly around the at least one functional unit and the connection end of the at least one electrode, with it preferably being formed by primary shaping around the functional unit and the connection end, in particular by moulding, such as, for example, injection moulding. For this purpose, it is possible to cast around or inject around the at least one functional unit and the respective connection end of the at least one electrode in a mould. In order to fix the individual components relative to one another during the moulding process, the mould can have appropriate recesses, bars, or the like. The stimulation end of the electrode and a substantial part of the electrode lead are arranged outside of the mould during the moulding process. The one-part design of the housing has the advantage that no further joining processes for connecting numerous housing parts are necessary. This also contributes to reducing the costs of the medical implant.

In another advantageous embodiment of the present invention, the housing comprises at least two housing parts. The at least two housing parts are produced, for example, by means of moulding, preferably by means of injection moulding, and the required functional units are then inserted into the “prefabricated” housing parts. The respective connection ends of the required electrodes can be joined to the corresponding functional unit before or after insertion into the mould, for example by means of a soldered connection. However, the housing can also comprise more than two housing parts, depending on production and/or use requirements. At least one housing part of the prefabricated housing preferably comprises an opening for passage of the electrode lead(s). Since the individual parts of the housing can be produced separately before insertion of the required components, the design of the mould is simplified since, for example, no outlet for guiding out electrode leads has to be provided.

On the inside of the housing, the housing parts preferably comprise at least one structure for assembly of the at least one functional unit. The structure for assembly of the at least one functional unit can comprise, for example, recesses, bars, frames, etc, which facilitate positioning during insertion as well as fixing of the different components in place in the housing. Furthermore, centring elements, for example indents or projections, can also be provided, which ensure that the individual parts of the housing are joined together with an accurate fit.

In the case of a multi-part design of the housing, the housing can, for example, be formed of two stainless steel or titanium half shells that are connected in a fluid-tight manner. To create a fluid-tight connection, the titanium half shells can, for example, first of all be fixed to one another in an exact position by means of a laser spot-welding process and the surrounding seam can then be welded.

However, the housing is preferably made of plastic. Plastic is suitable for producing both a one-part housing and a multi-part housing. The plastic is preferably a biocompatible plastic, such as, for example, polyurethane. Housings or housing parts that are made of plastic materials can be produced in a very inexpensive manner, for example by injection moulding. In the case of a multi-part plastic housing, the individual housing parts can be easily and inexpensively connected with one another, for example by gluing, preferably with epoxy, ultrasonic welding or thermal joining.

In order to meet the mechanical and electrical insulation requirements for medical implants, the wall thickness of the plastic housing should not go below a certain limit. When using polyurethane, the wall thickness is preferably at least 0.5 mm.

However, the housing can also comprise other biocompatible materials. For example, the housing can comprise a meshed fabric, referred to in the following as a “bug screen”. The bug screen preferably has a mesh width of approximately 1 mm. The mesh width is to be adjusted to the wavelength to be screened and should be approximately 1/10^{th} of this wavelength. Normal mesh widths used are between 0.5 and 5 mm. The bug screen is advantageously made of metal. If such a bug screen is used as the housing, it can have a one-part or multi-part design. In the case of a multi-part design, the individual housing parts are preferably joined to one another, for example by at least partial catching and/or wedging, or by welding/soldering of the respective edge areas of the individual housing parts. The use of a bug screen represents an inexpensive alternative to, for example, injection moulded plastic housings.

According to an advantageous development of the invention, the housing is provided at least in parts with an electrically conductive material. The electrically conductive material is arranged on the housing such that it has a shielding effect against electric interference fields, and the electromagnetic compatibility (EMC) is thus ensured. In the case of prefabricated housing parts, the electrically conductive material is preferably provided on the inside of the respective housing parts, whereas in a one-part housing, the electrically conductive material is preferably provided on the outside of the housing. If a bug screen made of an electrically conductive metal is used as the housing, an additional arrangement of an electrically conductive material is unnecessary. The housing can be provided with an electrically conductive material by means, for example, of metallisation or by coating with conductive plastics such as, for example, polythiophene.

In a further development of the medical implant according to the invention, the stimulation end of the electrode and at least a part of the electrode lead are arranged outside of the housing, and the housing and at least a section of the part of the electrode lead that is arranged outside of the housing have a sheathing. The sheathing ensures that the housing forms a fluid-tight unit, even in the region in which the electrode lead is guided out of the housing. In the case of a multi-part housing or the use of a bug screen as the housing, it is preferred for the entire housing to be sheathed. However, it is also taken into consideration to not sheathe the entire housing, but rather, for example, only that part of the housing which abuts the electrode lead. Partial sheathing of the housing is suitable, for example, if the housing is designed in one part. The housing of the medical implant preferably comprises, in particular in the case of just a partial sheathing, a biocompatible material such as, for example, titanium, polyurethane or silicone rubber.

The sheathing preferably comprises a biocompatible material such as, for example, silicone rubber. Silicone materials generally have a higher biocompatibility than, for example, stainless steel and, as compared to the use of metals, allow a further reduction in the costs and size of the medical implant. Furthermore, silicone materials allow injection around the housing, which, in terms of production technology, is easy to carry out and inexpensive. The layer thickness of the sheathing is generally at least 0.5 mm.

According to a preferred development of the present invention, the energy storage comprises an accumulator and/or a capacitor. The energy storage can hereby comprise either an accumulator or a capacitor, or a combination of an accumulator and a capacitor. However, the energy storage preferably comprises an accumulator or an accumulator/capacitor combination.

In the case of medical implants, lithium batteries that have an expected life time of approximately 10 years are normally used for supplying energy. The amount of charge that can be stored by a battery is referred to as “capacity” (nominal capacity) and has a corresponding influence on the size of the battery. In order to have such a long life time, the capacity of the battery is correspondingly large, which in turn results in battery dimensions that cannot be ignored. Thus, in conventional medical implants, the battery also takes up approximately ⅓ of the overall dimension of the medical implant.

However, according to the invention, a rechargeable accumulator is used instead of a battery. The accumulator preferably has a significantly smaller power reserve than conventionally used batteries, and can therefore also be produced with significantly smaller outer dimensions. The accumulator preferably has a power reserve of less than approximately four years, more preferred of less than approximately two years, and particularly preferred of approximately one year. Similar to the battery, the amount of charge that can be stored by an accumulator is specified in ampere hours (Ah) and referred to as capacity. In an accumulator having approximately one year of power reserve, the capacity is approximately 1 Ah. The capacity of the accumulator is preferably less than 300 mAh, more preferred less than 200 mAh, and most preferred 100 mAh or less. For a charging process with 3 V and 100 mAh, approximately 300 mW would be applied to charge the accumulator so that any tissue damage in the patient can be prevented. By reducing the capacity to approximately one year of power reserve, the accumulator could therefore be designed significantly smaller, which in turn contributes to reducing the overall dimension of the medical implant. It is additionally noted that the power reserve and/or period of charge of the accumulator is dependent on whether the control electronics have to emit pulses frequently or only rarely, and thus the reduction in size can be considerable depending on the field of use of the medical implant.

If the energy storage is designed in the form of an accumulator, a capacitor can also be additionally provided. In this case, the capacitor acts as a type of “emergency energy storage”, which prevents a failure or malfunctioning of the medical implant if the accumulator only has a very small charge left or is completely discharged. A warning function can be simultaneously emitted, which indicates to the wearer of the medical implant that it must be charged soon.

The energy storage is preferably designed to be recharged by means of high-frequency energy, magnetic-inductive charging, the heat of a patient, chemical energy, mechanical movement or lung movement. As regards charging by means of high-frequency energy, the accumulator could have a charging coil with a ferrite core, via which at a frequency above that of the pacemaker up to approximately 100 kHz can be charged in a contactless manner. Charging by means of the mechanical movement of the patient results from the movement of a patient's torso, for example when walking, running or bending over. Charging by means of lung movement (respiration) on the other hand only occurs as a result of the inherent movement of the lungs, without the patient having to move his body as a whole for this purpose. Furthermore, the energy storage could be designed to be charged by means of cardiac contractions, vasculature contractions (pulsating bloodstream) or fluid flow. A conversion of the heat of a patient into current or the conversion of the chemical energy of cells close to the site of implantation into electric energy is also taken into consideration. The possibilities for designing an accumulator that is to be recharged by means of high-frequency energy, magnetic-inductive changing, the heat of a patient, chemical energy, mechanical movement or lung movement are known from the prior art, and thus a more detailed explanation has not been provided at this point.

The control electronics preferably comprise an application-specific integrated circuit (ASIC). Owing to the design as an integrated circuit, the function of the control electronics cannot be manipulated, which is why the manufacturing costs for an ASIC are low. Furthermore, ASICs work very efficiently since their architecture is adapted to a specific problem, as a result of which a longer life time of the energy storage is ensured. Furthermore, they are readily available, resulting in comparatively lower costs. Thus, by providing an application-specific integrated circuit, the overall costs of the medical implant can be reduced and the life time of the energy storage can be increased.

According to another aspect of the present invention, a kit of parts is provided, which comprises at least two medical implants as specified above. The at least two medical implants have different designs of electrodes, in particular of the respective stimulation ends. According to the invention, complete medical implants are hereby provided, which differ as regards the design of the electrodes, in particular of the respective stimulation ends of the electrodes. In the prior art known to date, uniform medical implants were provided in each case, wherein different electrodes were inserted into the separate terminal housing depending on the purpose of use. However, in view of the significantly reduced manufacturing costs of the medical implant according to the invention, for example due to the omission of a separate terminal housing and special electric bushings, it is economically acceptable to also stock complete and/or ready-to-use medical implants, including the respective electrodes. Owing to the provision of a kit of parts, a selection of implants that are immediately ready for use can be made available to the treating physician for all common indications.

There are basically two types of electrodes, i.e. unipolar and bipolar electrodes. In the case of unipolar electrodes, the corresponding signal is only conducted in one direction whereas in bipolar electrodes it is conducted in two directions. The anchoring of the stimulation end of the electrode in the heart muscle can in turn be carried out actively, for example by fixing it in the muscle tissue using screw electrodes, or passively, for example by hooking it to the muscle fibres using anchor electrodes. A kit of parts comprising at least two medical implants with different designs of electrodes, in particular of the respective stimulation ends, is therefore particularly advantageous since ready-to-use medical implants with different electrodes for different purposes, depending on the positioning of the electrode in the heart and the illness of the patient, can be stocked at the same time.

### BRIEF DESCRIPTION OF THE DRAWINGS

FIG. 1 schematically shows a front view of a medical implant of the prior art.

FIG. 2 schematically shows a front view of a medical implant according to the present invention.

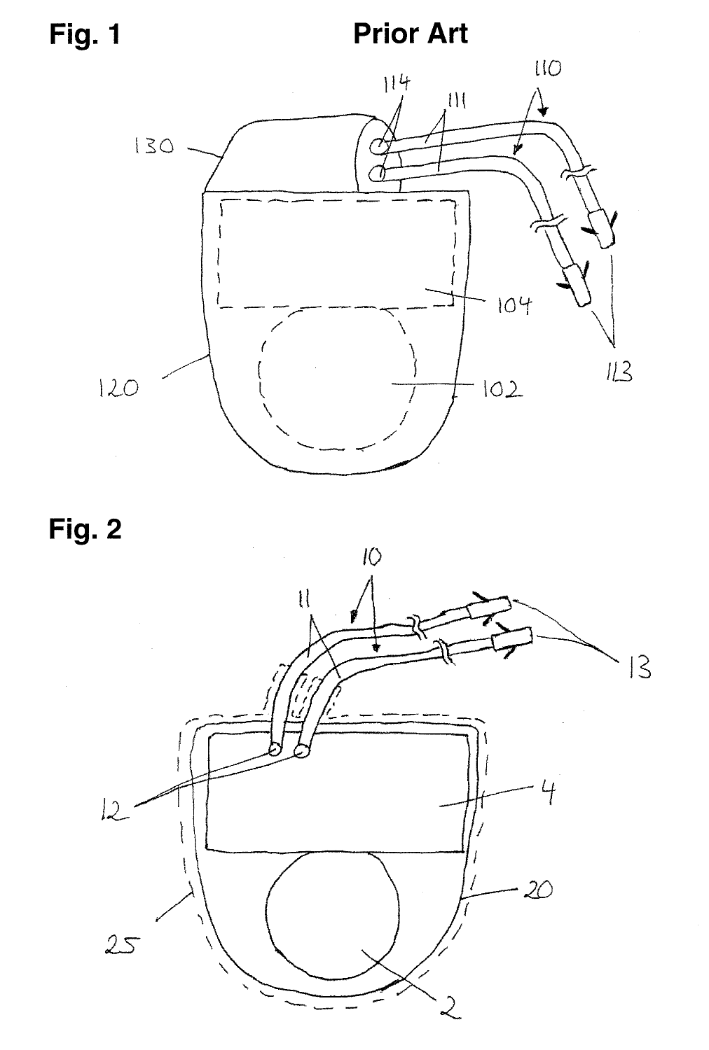

### DETAILED DESCRIPTION OF PREFERRED EMBODIMENTS

Preferred embodiments of the present invention are described in detail below with reference to the accompanying figure.

Schematically shown in FIG. 2 is a front view of a medical implant according to the present invention. The medical implant comprises a housing 20, in which control electronics 4 and an energy storage 2 are arranged. The energy storage 2 preferably comprises a rechargeable accumulator having a capacity that is sufficient for approximately one year of power reserve, as a result of which the accumulator can be designed significantly smaller than, for example, the energy storage 102 of the medical implant of the prior art. This small size of the accumulator 2 in turn contributes to a reduction of the overall dimension of the medical implant. However, the form of the accumulator 2 is not limited to the round form as shown in FIG. 2, and it can rather be designed in any suitable form, such as, for example, oval or rectangular.

The shown embodiment of the medical implant according to the invention furthermore comprises two electrodes 10, each having a connection end 12, an electrode lead 11 and a stimulation end 13 that is to be arranged on the heart muscle of the patient to be treated. In the embodiment shown in FIG. 2, the electrodes 10 are shown as so-called “anchor electrodes”, with the respective stimulation end 13 being hooked to the heart muscle of the patient. However, the type of design of the electrodes 10 is not limited to this form, and the electrodes 10 as well as their corresponding stimulation ends 13 can rather have any suitable form. The connection end 12 of the electrode 10 is directly connected in each case with the control electronics 4. Connection of the connection end 12 of the electrode 10 with the control electronics 4 can hereby be carried out, for example, by means of bonding or a soldered connection. By directly connecting the connection end 12 of the electrode 10 with the control electronics 4, it is possible to dispense with an additional terminal housing such as is necessary in medical implants of the prior art. Since such a terminal housing takes up approximately ⅓ of the overall dimension of a medical implant, the omission of this terminal housing leads to a significant reduction of the overall dimension of the medical implant.

As is apparent from FIG. 2, the entire housing 20 as well as a part of the electrode lead insulators 11 is provided with a sheathing 25. The electrode lead insulators 11 are preferably each provided with a sheathing having a length of approximately 1 cm to 10 cm. This length is sufficient for providing a fluid-tight seal between the housing 20 and the electrode leads 11, even in the case of permanent dynamic load. The respective electrode leads 11 can hereby be sheathed individually (as shown in FIG. 2), or as a unit together with the housing 20.

Even though the housing 20 shown in FIG. 2 is designed as one part, the housing 20 can also have a plurality of housing parts, which are sheathed together with the corresponding sections of the electrode leads 11 once the individual housing parts have been joined together. The sheathing 25 of the respective housing 20 takes place with a biocompatible material, preferably with a silicone rubber. In the case of a one-past housing 20, as shown in FIG. 2, the sheathing 25 can also take place just in the upper region of the housing 20 around the electrode leads 11, provided that a fluid-tight unit is created between the housing 20 and the electrode leads 11 lying outside of the housing 20.

However, the type of design of the medical implant is not limited to the embodiment shown in the figure, but rather also extends to modifications and variations within the scope of the following claims.

1. A cardiac implant, comprising:
at least one functional unit including an energy storage, control electronics or a combination thereof;
a housing for the at least one functional unit; and
at least one electrode having a connection end, an electrode lead and a stimulation end,
the connection end of the electrode being arranged in the housing and being attached directly to the functional unit.

2. A cardiac implant according to claim 1, wherein the housing is configured as a single part.

3. A cardiac implant according to claim 1, wherein the housing comprises at least two housing parts.

4. A cardiac implant according to claim 3, wherein the housing parts have on the inside of the housing at least one structure for assembly of the at least one functional unit.

5. A cardiac implant according to claim 1, wherein the housing is formed of plastic.

6. A cardiac implant according to claim 1, wherein the housing is provided at least in parts with an electrically conductive material.

7. A cardiac implant according to claim 1, wherein the connection end of the electrode is directly connected with the control electronics.

8. A cardiac implant according to claim 7, wherein the connection is carried out by bonding or includes a soldered connection.

9. A cardiac implant according to claim 1, wherein the stimulation end and at least a part of the electrode lead are arranged outside of the housing, and the housing and at least a section of the part of the electrode lead arranged outside of the housing have a sheathing.

10. A cardiac implant according to claim 9, wherein the sheathing comprises a biocompatible material.

11. A cardiac implant according to claim 1, wherein the energy storage comprises an accumulator and/or a capacitor.

12. A cardiac implant according to claim 11, wherein the energy storage is designed to be recharged by high-frequency energy, magnetic-inductive charging, the heat of a patient, chemical energy, mechanical movement or lung movement.

13. A cardiac implant according to claim 1, wherein the control electronics comprise an application-specific integrated circuit.

14. A kit of parts, comprising at least two cardiac implants according to claim 1.

15. A kit of parts according to claim 14, wherein the at least two cardiac implants comprise different designs of the respective stimulation ends.

16. A cardiac implant according to claim 1, wherein the connection end is attached directly to the functional unit by bonding or a soldered connection.

17. A cardiac implant according to claim 5, wherein the housing is formed of polyurethane.

18. A cardiac implant according to claim 10, wherein the sheathing comprises silicone rubber.

19. A cardiac implant according to claim 1, wherein the cardiac implant is a pacemaker, a cardioverter, or a defibrillator.
